(19) United States
(12) Patent Application Publication
(10) Pub. No.: US 2022/0022830 A1
(43) Pub. Date: Jan. 27, 2022

(54) X-Ray Apparatus And Patient Instruction System

(71) Applicant: Jairo Ochoa- Madrigal, Hughson, CA (US)

(72) Inventor: Jairo Ochoa- Madrigal, Hughson, CA (US)

(21) Appl. No.: 16/935,621

(22) Filed: Jul. 22, 2020

(51) Int. Cl. A61B 6/00; G06F 40/58

(52) U.S. Cl. CPC A61B 6/46; G06F 40/58; A61B 6/40; A61B 6/542; A61B 6/52

(57) ABSTRACT

An X-ray apparatus and patient instruction system for easily instructing patients through an X-ray procedure in a variety of languages includes an X-ray apparatus, a computer, and a controller. The X-ray apparatus comprises an X-ray generator coupled within an X-ray housing to create a digital X-ray image. An X-ray CPU and a speaker are coupled within the X-ray housing. The computer comprises a computer CPU coupled within the computer housing. The computer CPU is in operational communication with the X-ray CPU stores a plurality of audio recordings of X-ray procedure instructions to play through the speaker. A computer display shows the digital X-ray images from the X-ray generator. The controller comprises a control button coupled to the controller handle to operate the speaker and the X-ray generator.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

The disclosure relates to X-ray devices and more particularly pertains to a new X-ray device for easily instructing patients through an X-ray procedure in a variety of languages.

### (2) Description of Related Art Including Information Disclosed Under 37 CFR 1.97 and 1.98

The prior art relates to X-ray devices and medical technologies with preprogrammed audio instructions. Existing devices often incorporate wearable medical devices or only serve to translate instructions. Known devices are not incorporated with X-ray technology and do not serve to integrate a series of instructions within an X-ray imaging procedure.

### BRIEF SUMMARY OF THE INVENTION

An embodiment of the disclosure meets the needs presented above by generally comprising an X-ray apparatus, a computer, and a controller. The X-ray apparatus comprises an X-ray generator coupled within an X-ray housing. The X-ray generator creates a digital X-ray image. An X-ray CPU is coupled within the X-ray housing and is in operational communication with the X-ray generator. A speaker is coupled within the X-ray housing and is in operational communication with the X-ray CPU. The computer comprises a computer housing and a computer CPU coupled within the computer housing. The computer CPU is in operational communication with the X-ray CPU stores a plurality of audio recordings of X-ray procedure instructions to play through the speaker of the X-ray apparatus. A computer display is coupled within the computer housing and is in operational communication with the computer CPU to show digital X-ray images from the X-ray generator. The controller comprises a controller handle and a control button coupled to the controller handle. The control button is in operational communication with the X-ray CPU and the computer CPU.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is an isometric view of an X-ray apparatus and patient instruction system according to an embodiment of the disclosure.

FIG. 2 is a side elevation in-use view of an embodiment of the disclosure.

FIG. 3 is a block diagram of an embodiment of the disclosure.

### DETAILED DESCRIPTION OF THE INVENTION

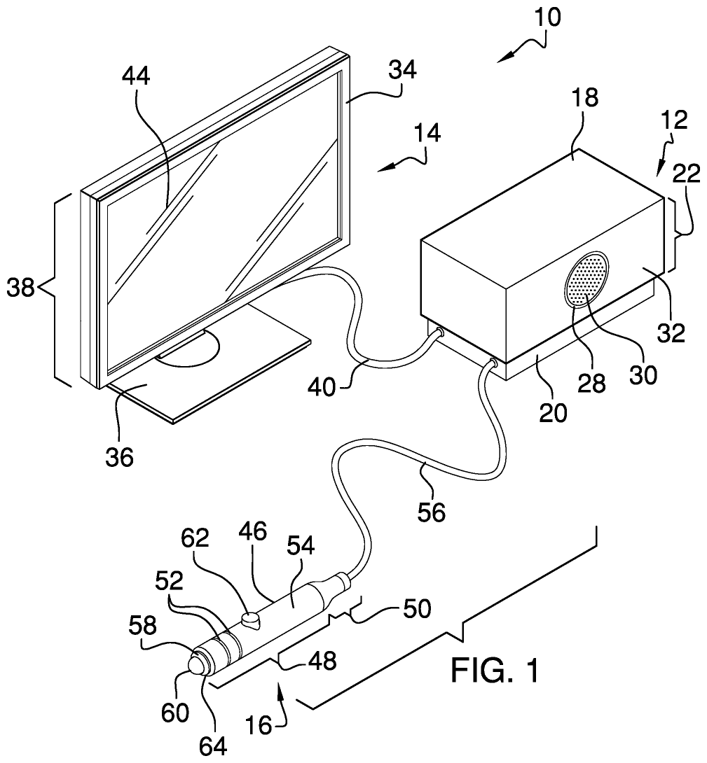

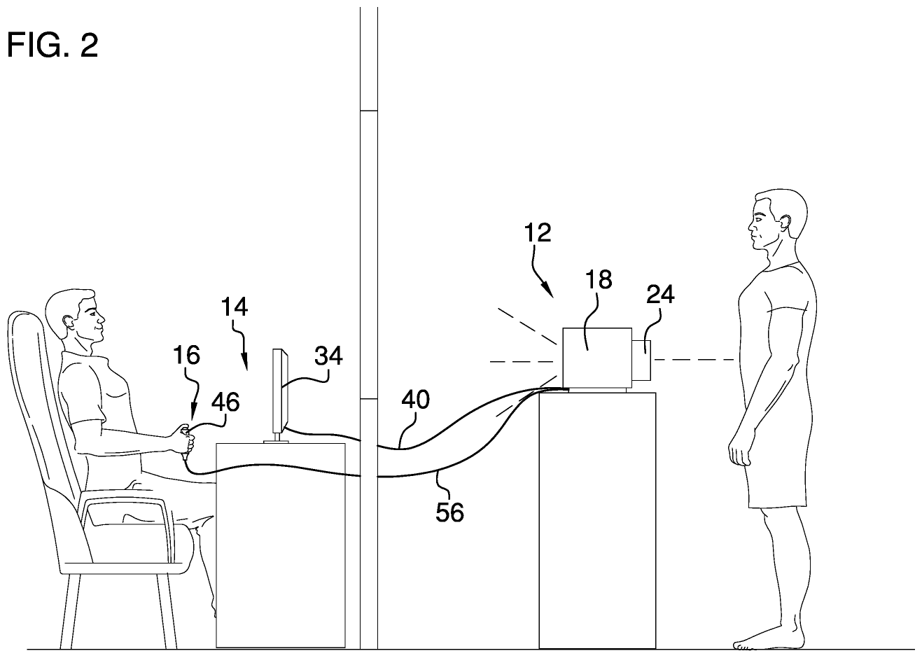

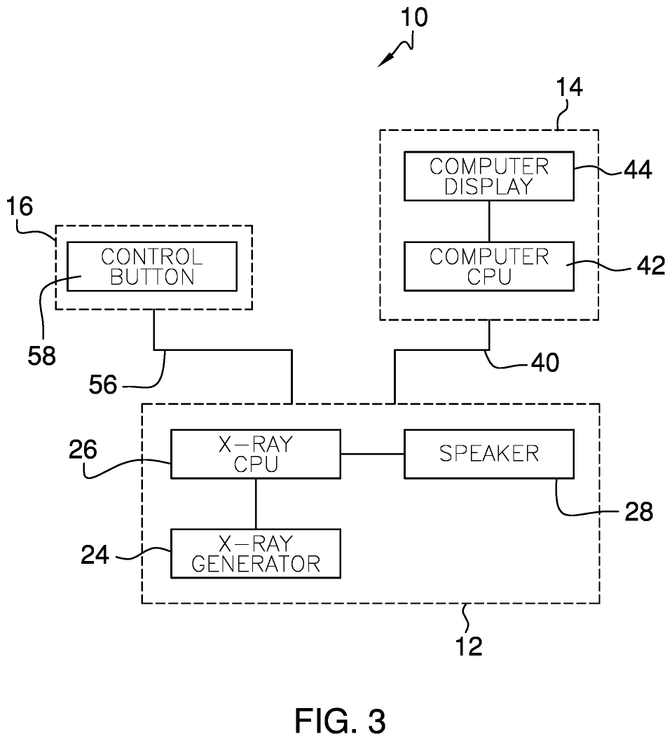

With reference now to the drawings, and in particular to FIGS. 1 through 3 thereof, a new X-ray device embodying the principles and concepts of an embodiment of the disclosure and generally designated by the reference numeral 10 will be described.

As best illustrated in FIGS. 1 through 3, the X-ray apparatus and patient instruction system 10 generally comprises an X-ray apparatus 12, a computer 14, and a controller 16. The X-ray apparatus 12 comprises an X-ray housing 18. The X-ray housing 18 may have a lower housing base portion 20 and a rectangular prismatic upper housing portion 22. An X-ray generator 24 is coupled within the X-ray housing 18. The X-ray generator 24 creates a digital X-ray image. An X-ray CPU 26 is coupled within the X-ray housing 18. The X-ray CPU 26 is in operational communication with the X-ray generator 24. A speaker 28 is coupled within the X-ray housing 18. The speaker 28 may have a speaker grille 30 extending through a front face 32 of the upper housing portion 22. The speaker 28 is in operational communication with the X-ray CPU 26.

The computer 14 comprises a computer housing 34. The computer housing 34 may have a base portion 36 and a monitor portion 38. The base portion 36 may be rectangular prismatic. A computer connection wire 40 extends from the computer housing 34 to the X-ray housing 18.

A computer CPU 42 is coupled within the computer housing 34. The computer CPU 42 is in operational communication with the X-ray CPU 26 and stores a plurality of audio recordings of X-ray procedure instructions to play through the speaker 28 of the X-ray apparatus. A computer display 44 is coupled within the computer housing 34. The computer display 44 is in operational communication with the computer CPU 42 to show digital X-ray images from the X-ray generator 24.

The controller 16 comprises a controller handle 46. The controller handle 46 may have a cylindrical portion 48 and a tapered bottom portion 50 for improved ergonomics. The controller handle 46 may have a pair of grip rings 52 extending around a sidewall 54 of the cylindrical portion for slip prevention. There may be a controller connection wire 56 extending from the tapered bottom portion 50 to the X-ray housing 18. A control button 58 is coupled to the controller handle 46. The control button 58 may include a top button 60 and a side button 62. The top button 60 may be coupled to a top end 64 of the cylindrical portion and the side button 62 may be coupled through the sidewall 54 of the cylindrical portion. The control button 58 is in operational communication with the X-ray CPU 26 and the computer CPU 42. The control button 58 activates the speaker 28 to reproduce one step of the X-ray procedure instructions and activates a rotor stage of the X-ray generator 24 to prepare an exposure tube 60 of the X-ray generator for the language and protocol selected on the computer 14. The control button 58 then activates the X-ray generator 25 to create the digital X-ray image. The control button 58 may alternate between these functions with each depression or may split these functions between the top button 60 and the side button 62.

In use, the desired language and X-ray protocol are selected on the computer 14. The technician then uses the controller 16 to prompt the patient with an auditory instruction via the speaker 28, such as how to position their body or breathe, and then to create a digital X-ray image to be shown on the computer display 44.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. An X-ray apparatus and patient instruction system comprising:
an X-ray apparatus comprising:
an X-ray housing;
an X-ray generator coupled within the X-ray housing, the X-ray generator creating a digital X-ray image;
an X-ray CPU coupled within the X-ray housing, the X-ray CPU being in operational communication with the X-ray generator; and
a speaker coupled within the X-ray housing, the speaker being in operational communication with the X-ray CPU;
a computer comprising:
a computer housing;
a computer CPU coupled within the computer housing, the computer CPU being in operational communication with the X-ray CPU and storing a plurality of audio recordings of X-ray procedure instructions to play through the speaker of the X-ray apparatus; and
a computer display coupled within the computer housing, the computer display being in operational communication with the computer CPU to show digital X-ray images from the X-ray generator; and
a controller comprising:
a controller handle; and
a control button coupled to the controller handle, the control button being in operational communication with the X-ray CPU and the computer CPU.

2. The X-ray apparatus and patient instruction system of claim 1 further comprising the control button activating the speaker to reproduce one step of the X-ray procedure instructions and activating a rotor stage of the X-ray generator to prepare an exposure tube of the X-ray generator for the language and protocol selected on the computer, the control button then activating the X-ray generator to create the digital X-ray image.

3. The X-ray apparatus and patient instruction system of claim 1 further comprising the X-ray housing having a lower housing base portion and a rectangular prismatic upper housing portion; the speaker having a speaker grille extending through a front face of the upper housing portion.

4. The X-ray apparatus and patient instruction system of claim 1 further comprising the controller handle having a cylindrical portion and a tapered bottom portion, a top button of the control button being coupled to a top end of the cylindrical portion, a side button of the control button being coupled through a sidewall of the cylindrical portion.

5. The X-ray apparatus and patient instruction system of claim 4 further comprising the controller having a controller connection wire extending from the tapered bottom portion to the X-ray housing; the computer having a computer connection wire extending from the computer housing to the X-ray housing.

6. The X-ray apparatus and patient instruction system of claim 4 further comprising the controller handle having a pair of grip rings extending around the sidewall of the cylindrical portion.

7. An X-ray apparatus and patient instruction system comprising:
an X-ray apparatus comprising:
an X-ray housing, the X-ray housing having a lower housing base portion and a rectangular prismatic upper housing portion;
an X-ray generator coupled within the X-ray housing, the X-ray generator creating a digital X-ray image;
an X-ray CPU coupled within the X-ray housing, the X-ray CPU being in operational communication with the X-ray generator; and
a speaker coupled within the X-ray housing, the speaker having a speaker grille extending through a front face of the upper housing portion, the speaker being in operational communication with the X-ray CPU;
a computer comprising:
a computer housing;
a computer connection wire extending from the computer housing to the X-ray housing;
a computer CPU coupled within the computer housing, the computer CPU being in operational communication with the X-ray CPU and storing a plurality of audio recordings of X-ray procedure instructions to play through the speaker of the X-ray apparatus; and
a computer display coupled within the computer housing, the computer display being in operational communication with the computer CPU to show digital X-ray images from the X-ray generator; and
a controller comprising:
a controller handle, the controller handle having a cylindrical portion and a tapered bottom portion, the controller handle having a pair of grip rings extending around a sidewall of the cylindrical portion;
a controller connection wire extending from the tapered bottom portion to the X-ray housing; and
a control button coupled to the controller handle, a top button of the control button being coupled to a top end of the cylindrical portion, a side button of the control button being coupled through the sidewall of the cylindrical portion, the control button being in operational communication with the X-ray CPU and the computer CPU, the control button activating the speaker to reproduce one step of the X-ray procedure instructions and activating a rotor stage of the X-ray generator to prepare an exposure tube of the X-ray generator for the language and protocol selected on the computer, the control button then activating the X-ray generator to create the digital X-ray image.
